(19) United States
(12) Patent Application Publication
(10) Pub. No.: US 2021/0322480 A1
(43) Pub. Date: Oct. 21, 2021

(54) Platelet-Rich Plasma Compositions and Related Methods

(71) Applicant: BRAHM HOLDINGS, LLC, Germantown, TN (US)

(72) Inventor: Timothy R. BRAHM, Germantown, TN (US)

(21) Appl. No.: 17/235,166

(22) Filed: Apr. 20, 2021

Related U.S. Application Data
(60) Provisional application No. 63/012,325, filed on Apr. 20, 2020.

(51) Int. Cl. A61K 35/19; A61K 35/16; C07K 16/10

(52) U.S. Cl. CPC A61K 35/19; A61K 2039/505; C07K 16/10; A61K 35/16

(57) ABSTRACT

An antiviral composition is provided. The antiviral composition includes a platelet-rich plasma composition. Methods of treatment and initiating passive immunity for viral infections are also provided.

### CROSS-REFERENCE TO RELATED APPLICATIONS

The present application claims priority to U.S. Provisional Application No. 63/012,325, filed Apr. 20, 2020, the contents of which are incorporated in its entirety.

### BACKGROUND OF INVENTION

Viruses and the associated infections continue to pose a major medical risk and threat the safety and security of humanity. Pandemics associated with Ebola, SARS, West Nile and COVID-19 continue to pose such a threat. Methods of treatment and prevention are in desperate need.

Platelet-rich plasma is an autologous concentrate of platelets in a small volume of plasma. Platelet-rich plasma is useful in the treatment for bone, muscle, tendon, and cartilage injury. The healing potential of platelet-rich plasma preparations is largely attributed to its dense concentration of growth factors and cytokines derived from platelets. Multiple studies have demonstrated the role of platelet-rich plasma in accelerating and facilitating improved response to injury. The theoretical basis for the use of platelet-rich plasma in tissue repair is that large numbers of platelets appear immediately at the site of tissue injury and release growth factors and cytokines, thereby initiating the wound healing process.

Antibodies are found in blood plasma and can fight or otherwise neutralize any substance that may be introduced as a foreign body. Antibodies may also be released in response to a viral infection and remain in a human's blood plasma to prevent against future infections.

Platelet-rich plasma can include white blood cells. Such white blood cells include neutrophils, eosinophils, basophils, monocytes and lymphocytes. Lymphocytes are subdivided into T cells, B cells and NK cells. The B cells have the capability of secreting antibodies in response to infection. Antibodies are small proteins specific for infectious agents that the body has encountered before.

### SUMMARY OF INVENTION

An antiviral composition is provided. The method includes a platelet-rich plasma composition and, optionally, a pharmaceutically acceptable carrier. The platelet-rich plasma composition includes platelet-rich plasma derived from blood of a mammal that has active immunity to one or more viral infections. According to one embodiment, the mammal is a horse, pig, or cow. According to one embodiment, the mammal is a human. According to one embodiment, the platelet-rich plasma is derived from umbilical cord blood. According to one embodiment, the platelet-rich plasma composition includes one or more antibodies therapeutically effective against one or more viruses. According to one embodiment, the platelet-rich plasma composition includes one or more antibodies obtained from the immunoglobulin-containing serum of a human that has active immunity to one or more viral infections. According to one embodiment, the one or more viral infections is caused by one or more viruses that results in a respiratory infection. According to one embodiment, the one or more viruses is a severe acute respiratory syndrome (SARS) virus. According to one embodiment, the SARS virus is a coronavirus. According to one embodiment, the SARS virus is SARS-CoV-2 or a variant thereof. According to one embodiment, the one or more viruses is an RNA virus in that the genetic material for the virus is encoded in ribonucleic acid.

A method of treating a viral infection in a patient in need of treatment is also provided. The method includes the step of administering an effective amount of the antiviral composition as provided herein. Upon administration, one or more symptoms associated with the viral infection in the patient in need in need of treatment are alleviated. Administration may also neutralize, kill or eradicate the viral infection.

A method of initiating passive immunity to a viral infection to a patient in need of passive immunity is also provided. The method includes the step of administering an effective amount of the antiviral composition as provided herein. Upon administration, the patient acquires passive immunity to the viral infection.

### BRIEF DESCRIPTION OF THE DRAWINGS

FIG. 1 illustrates one embodiment of a method of preparing an antiviral composition.

### DETAILED DESCRIPTION OF THE INVENTION

The present disclosure will now be described more fully hereinafter with reference to exemplary embodiments thereof. These exemplary embodiments are described so that this disclosure will be thorough and complete, and will fully convey the scope of the disclosure to those skilled in the art. Indeed, the present disclosure may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise. As used in the specification, and in the appended claims, the words “optional” or “optionally” mean that the subsequently described event or circumstance can or cannot occur. For example, the phrase “optionally soaking the membrane” means that the soaking step may or may not be performed.

As used herein the term “virus” refers to any virus that infects the human body. Such a virus can cause manifest as a respiratory infection. The viruses contemplated herein include, but are not limited to, a severe acute respiratory syndrome (SARS) virus. The SARS virus include, but is not limited to, a coronavirus such as, for example, a COVID-19 type virus.

As used herein, the term “antiviral” refers to the ability to kill a virus or suppress the virus' ability to replicate and multiply.

As used herein, the term “platelet-rich plasma” refers to a human plasma concentrate rich in platelets as well as white blood cells and is prepared by any of the preparation techniques known to those skilled in the art. The platelet-rich plasma as provided herein is obtained from a human that has active immunity to one or more viral infections.

As used herein, the term “effective amount” refers to an amount of a particular composition sufficient to elicit the desired therapeutic effects.

As used herein, the term “pharmaceutically-acceptable carrier” as used herein refers a pharmaceutically-acceptable material, composition or vehicle, such as a liquid or diluent involved in carrying or transporting the platelet-rich plasma composition.

As used herein, the term “acceptable” refers to the sense of being compatible with any the components of the antiviral composition and not injurious to the patient.

As used herein, the term “active immunity” refers to the state of a patient that has acquired immunity to a viral infection either by contracting the viral infection (or being exposed to the virus) thereby causing the body to produce viral-specific antibodies that are found in platelet-rich plasma. Active immunity may allow for protection from the viral infection for the duration of at least three months, at least six months, at least one year, or a lifetime.

As used herein, the term “vaccine-induced immunity” refers to the state of a patient that has acquired immunity to a viral infection after administration of a vaccine.

An antiviral composition is provided herein. The antiviral composition is particular useful in the treatment of viral infections, prevention of viral infections, as well as initiating passive immunity to populations that might be susceptive to viral infections. According to particular embodiment, the antiviral composition as provided herein is administered in a prophylactic manner to prevent infection in high-risk cases, such as vulnerable individuals with underlying medical conditions, health care providers, and individuals with exposure to confirmed cases of a viral infection. According to one embodiment, the antiviral composition may be used to treat individuals with clinical disease to reduce symptoms and mortality. According to one embodiment, the antiviral composition may be used to reduce symptoms associated with an upper respiratory illness such as cough, inflammation, asthma or emphysema.

The antiviral composition as provided herein includes a platelet-rich plasma composition. According to one embodiment, the platelet-rich plasma composition includes a therapeutically amount of platelet-rich plasma. The platelet-rich plasma may be derived from mammalian blood. The mammal may be a human, horse, cow or pig. According to one embodiment, the platelet-rich plasma may be derived from human blood and utilized as provided herein in an autologous manner. According to one embodiment, the platelet-rich plasma may be derived from human blood and utilized as provided herein in an allogenic manner. When utilized in an allogenic manner, blood from one or more (or a plurality) of human blood donors may be pooled or otherwise mixed in large batch. According to an alternative embodiment, the platelet-rich plasma may be derived from a mammal other than a human.

The human blood that is the source of platelet-rich plasmas may be obtained from a human that has active immunity or vaccine-induced immunity to one or more viral infections. According to one embodiment, the human blood is umbilical cord blood from a female mammal that has active immunity or vaccine-induced immunity to one or more viral infections at the time of pregnancy or delivery of an infant. According to one embodiment, the human blood is umbilical cord blood from a female human that has active immunity or vaccine-induced immunity to one or more viral infections at the time of pregnancy or delivery of an infant. According to one embodiment, the blood is umbilical cord blood from a female mammal that has active immunity or vaccine-induced immunity to one or more viral infections at the time of pregnancy or delivery of an infant and does not contain blood from another source in the human body (i.e., not obtained by typical blood donations via intravenous delivery).

The platelet-rich composition as provided herein includes a therapeutically active amount of white blood cells as the platelets are not totally isolated from the other blood components. The white blood cells include one or more B cells that produce one or more antibodies that may introduce passive immunity to viral infections.

According to one embodiment, the platelet-rich plasma composition may include one or more antibodies obtained from the immunoglobulin-containing human convalescent serum of a human that has recovered from an infection caused by one or more viruses or otherwise has active immunity from one or more viral infections.

According to one embodiment, the platelet-rich plasma composition may include any one or more components that increase the efficacy of the antiviral composition or increase the efficiency of the immune system of a patient such as, for example, one or more vitamins, including, but not limited, vitamin A, vitamin B, vitamin C, vitamin D, and vitamin E.

According to one embodiment, the platelet-rich plasma composition may include any one or more platelet-derived growth factor (PDGF), platelet-derived angiogenesis factor (PDAF), vascular endothelial growth factor (VEGF), platelet-derived epidermal growth factor (PDEGF), platelet factor 4 (PF-4), transforming growth factor beta (TGF-B or TGF-B1), acidic fibroblast growth factor (FGF-A), basic fibroblast growth factor (FGF-B), transforming growth factor alpha (TGF-A), insulin-like growth factors 1 and 2 (IGF-1 and IGF-2), beta-thromboglobulin-related proteins (BTG), thrombospondin (TSP), fibronectin, von Wallinbrand's factor (vWF), fibropeptide A, fibrinogen, albumin, plasminogen activator inhibitor 1 (PAI-1), osteonectin, regulated upon activation normal T cell expressed and presumably secreted (RANTES), vitronectin, fibrin D-dimer, factor V, antithrombin III, immunoglobulin-G (IgG), immunoglobulin-M (IgM), immunoglobulin-A (IgA), a2-macroglobulin, angiogenin, Fg-D, elastase, keratinocyte growth factor (KGF), epidermal growth factor (EGF), fibroblast growth factor (FGF), tumor necrosis factor (TNF), fibroblast growth factor (FGF) and interleukin-1 (IL-1), Keratinocyte Growth Factor-2 (KGF-2), and combinations thereof.

According to one embodiment, the platelet-rich plasma composition may include one or more bioactive agents such as, for example, physiologically compatible minerals, growth factors, antibiotics, chemotherapeutic agents, antigen, antibodies, enzymes, vectors for gene delivery and hormones may also be added to the antiviral composition.

According to one embodiment, the platelet-rich plasma composition may include one or more pesticides such as ivermectin, avermectin, or abamectin. According to one embodiment, the platelet-rich plasma composition may include one or more antibiotics.

According to one embodiment, the antiviral composition may include a carrier. The carrier may be any pharmaceutically acceptable carrier suitable for the mode of administration. According to one embodiment, the carrier may be a Ringer's lactate solution that includes a mixture of sodium chloride, sodium lactate, potassium chloride, and calcium chloride in water. According to one embodiment, the carrier includes a sterile saline solution. According to one embodiment, the carrier includes acetated Ringer's solution, phosphate buffered solution, TRIS-buffered saline solution, Hank's balanced salt solution, Earle's balanced salt solution, standard saline citrate, HEPES-buffered saline, dextrose, or glucose.

The amount of platelet-rich plasma composition and any carrier may vary depending upon the chosen carrier, the frequency of administration, and the severity or type of viral infection to be treated. According to one embodiment, the antiviral composition includes from typically about 0.1% to about 99.9% platelet-rich plasma composition with the balance optionally being any pharmaceutically acceptable carrier.

### Methods of Preparation

A method of preparing an antiviral composition is provided. Various techniques known in the art may be utilized for preparing the platelet-rich plasma composition portion of the antiviral composition as provided herein.

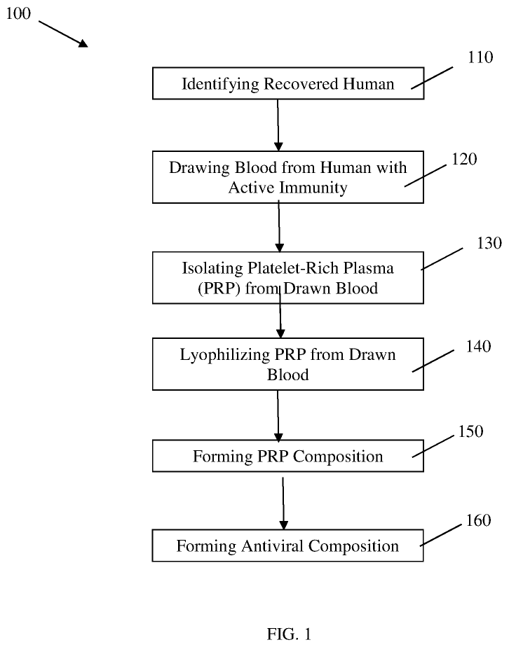

One embodiment of a method of preparing an antiviral composition is provided in FIG. 1. The method (100) includes the step of identifying a human that has active immunity (110). According to one embodiment, the human has recovered from a viral infection. According to one embodiment, the human has been treated with a vaccine. The step of identifying such a patient (110) may be performed by a physician or other medical professional that assisted a patient in recovery and can confirm active immunity. The step of identifying a patient with active immunity (110) may be via voluntary action by the patient to come forward and donate blood.

The method further includes the step of drawing or otherwise collecting blood from the human with active immunity (120). Upon collection, infectious disease testing of donor blood specimens may be performed for each tissue donor on a specimen collected at the time of donation or within seven days prior to or after donation. Advantageously, the methods that are used to screen for a communicable disease follow the regulations as set forth by the Federal Drug Administration and the American Association of Tissue Banks. Exemplary infectious disease testing includes, but is not limited to, antibodies to the human immunodeficiency virus, type 1 and type 2 (anti-HIV-1 and anti-HIV-2); nucleic acid test (NAT) for HIV-1; hepatitis B surface antigen (HBsAg); total antibodies to hepatitis B core antigen (anti-HBc—total, meaning IgG and IgM); antibodies to the hepatitis C virus (anti-HCV); NAT for HCV; antibodies to human T-lymphotropic virus type I and type II (anti-HTLV-I and anti-HTLV-II); and syphilis (a non-treponemal or treponemal-specific assay may be performed).

According to one embodiment, the drawn blood may be optionally screened for virus-neutralizing antibodies. According to one embodiment, following identification of those with high titers of neutralizing antibody, a serum containing these virus-neutralizing antibodies can be prepared and added to the platelet-rich plasma composition as provided herein.

Following collection, the blood may be cryopreserved by any methods known to those skilled in the art. For example, the blood may be cryopreserved in the liquid phase of liquid nitrogen. In one embodiment, the cryoprotectant is one commonly used in the industry, such as, for example, dimethyl sulfoxide (DMSO). In another embodiment, the cryoprotectant is a commercially available cryoprotectant such as Synth-a-Freeze® available from Invitrogen. Any cryoprotectant specific to blood described herein may be used. In one embodiment, cryopreservation is achieved using a controlled rate freezer, resulting in a 1° C. rate from nucleation to −35° C. and a 10° C. per minute cooling rate to a −90° C. end temperature, however, any cryopreservation method commonly known in the art may be used. According to one aspect, the blood remains cryopreserved until just prior to use or need.

The method further includes the step of isolating platelet-rich plasma from the drawn blood (130). According to one embodiment, the platelets of the platelet-rich plasma composition are separated from the red blood cells of whole blood, primarily through differential centrifugation, although any suitable method for separating platelets from whole blood may be employed in practicing this invention. According to a particular embodiment, the platelet-rich plasma is isolated using a commercially available platelet separation system. As such, the determination of which platelet separation system to use is largely dependent on the end-user (i.e., the medical professional administering the preparation). In an alternate embodiment, the platelet-rich plasma is isolated shortly after collection and remains cryopreserved until just prior to use. Currently, commercial platelet separation systems are available, many of which vary significantly in the relative amounts of platelets, leukocytes, erythrocytes, and anabolic and catabolic growth factors. Any commercially available or literature-based method of isolating platelets may be used (e.g., double-spin method; see de Mos M, van der Windt A E, Jahr H, et al. Can platelet-rich plasma enhance tendon repair. A cell culture study. Am J Sports Med. 2008; 36(6):1171-1178). Following collection of the platelet-rich plasma, the platelet-rich plasma may be cryopreserved by any methods known to those skilled in the art and as provided herein. Before use, the platelet-rich plasma may optionally be thawed.

The method further includes the step of lyophilizing the platelet-rich plasma (140). Lyophilization may be carried out by any acceptable commercial means. The step of lyophilizing the platelet-rich plasma may be carried out until a freeze-dried platelet-rich plasma composition results. According to one embodiment, the step of lyophilizing the platelet-rich plasma may be carried out by the methods set forth in U.S. Pat. No. 8,821,858, the contents of which are incorporated herein by reference.

The method further includes the step of forming the platelet-rich plasma composition (150). According to one embodiment, the formation step optionally includes re-hydrating the platelet-rich plasma composition with water. According to one embodiment, the water is de-ionized water.

The method further includes the step of forming the antiviral composition (160). According to one embodiment, the antiviral composition formation step optionally includes admixing the platelet-rich plasma with any pharmaceutically acceptable carrier. According to one embodiment, the platelet-rich plasma composition and any pharmaceutically acceptable carrier may be mixed or blended according to a variety of conventional techniques to form the antiviral composition as provided herein.

### Methods of Treatment and Prevention

Methods of treating a viral infection are provided. The method includes administering the antiviral composition as provided herein to a patient in need of treatment. Such a patient in need of treatment may be suffering from a viral infection caused by a virus as described herein.

Methods of reducing antiviral symptoms and mortality are provided. The method includes administering the antiviral composition as provided herein to a patient in need of treatment. Such a patient in need of treatment may be suffering from a viral infection caused by a virus as described herein and exhibiting one or more symptoms or a risk of death.

Methods of preventing a viral infection are provided. The method includes administering the antiviral composition as provided herein to a patient in need of treatment. The antiviral composition can be administered in a prophylactic manner to prevent infection in the general population. The antiviral composition can be administered in a prophylactic manner to prevent infection in high-risk cases, such as vulnerable individuals with underlying medical conditions, health care providers, and individuals with exposure to confirmed cases of an antiviral infection.

Methods of implementing passive immunity to a viral infection are provided. The method includes administering the antiviral composition as provided herein to a patient in need of treatment. The antiviral composition can be administered in a prophylactic manner to prevent infection in the general population.

Methods of supplementing herd or community immunity to a viral infection are provided. The method includes administering the antiviral composition as provided herein to a patient in need of treatment. The antiviral composition can be administered in a prophylactic manner to prevent infection in the general population.

According to one embodiment, the antiviral compositions described herein may be administered by a user (i.e., medical professional) according to any medically acceptable manner which include, but are not limited to, intramuscular, subcutaneous, intraperitoneal, percutaneous, soft tissue injection, intravenous, intravascular, intracerebral, transdermal, intraocular, topical, mucosal, or via inhalation directly to the lungs (e.g., through a nebulizer). Multiple administrations may occur essentially at the same time or separated in time. The mode of administration, the dosage administered, and the dosage number will vary per individual depending on a variety of factors, including pharmacokinetic properties, patient conditions and characteristics, extent of symptoms, concurrent treatments, frequency of treatment and the desired effect.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. An antiviral composition comprising:
a platelet-rich plasma composition comprising platelet-rich plasma derived from blood of at least one mammal, the one at least one mammal having active immunity to one or more viral infections; and
optionally, a pharmaceutically acceptable carrier.

2. The antiviral composition of claim 1, wherein the platelet-rich plasma composition comprises one or more antibodies therapeutically effective against one or more viruses.

3. The antiviral composition of claim 1, wherein the platelet-rich plasma composition comprises one or more antibodies obtained from the immunoglobulin-containing serum of a mammal that has active immunity to one or more viral infections.

4. The antiviral composition of claim 1, wherein the one or more viral infections is caused by one or more viruses that results in a respiratory infection.

5. The antiviral composition of claim 4, wherein the one or more viruses is a severe acute respiratory syndrome (SARS) virus.

6. The antiviral composition of claim 5, wherein the SARS virus is a coronavirus.

7. The antiviral composition of claim 1, wherein the mammal is a horse, pig, or cow.

8. The antiviral composition of claim 1, wherein the mammal is a human.

9. The antiviral composition of claim 1, wherein the platelet-rich plasma s derived from umbilical cord blood.

10. A method of treating a viral infection in a patient in need of treatment, the method including the step of:
administering an effective amount of the antiviral composition of claim 1,
wherein, upon administration, one or more symptoms associated with the viral infection in the patient in need in need of treatment are alleviated.

11. The method of claim 10, wherein the one or more viral infections is caused by one or more viruses that results in a respiratory infection.

12. The method of claim 11, wherein the one or more viruses is a severe acute respiratory syndrome (SARS) virus.

13. The method of claim 12, wherein the SARS virus is a coronavirus.

14. A method of initiating passive immunity to a viral infection to a patient in need of passive immunity, the method including the step of:
administering an effective amount of the antiviral composition of claim 1,
wherein, upon administration, the patient acquires passive immunity to the viral infection.

15. The method of claim 14, wherein the one or more viral infections is caused by one or more viruses that results in a respiratory infection.

16. The method of claim 15, wherein the one or more viruses is a severe acute respiratory syndrome (SARS) virus.

17. The method of claim 16, wherein the SARS virus is a coronavirus.
